(19) United States
(12) Patent Application Publication
(10) Pub. No.: US 2013/0282007 A1
(43) Pub. Date: Oct. 24, 2013

(54) MODULAR CATHETER

(71) Applicants: Evan Chong, South Strathfield (AU); Roman Greifeneder, Kyle bay (AU)

(72) Inventors: Evan Chong, South Strathfield (AU); Roman Greifeneder, Kyle bay (AU)

(73) Assignee: CATHRX LTD, NEW SOUTH WALES (AU)

(21) Appl. No.: 13/997,980

(22) PCT Filed: Dec. 23, 2011
(86) PCT No.: PCT/AU11/01677

§ 371 Date: Jun. 25, 2013

Related U.S. Application Data
(60) Provisional application No. 61/427,335, filed on Dec. 27, 2010.

(51) Int. Cl. A61B 18/14

(52) U.S. Cl. CPC A61B 18/1492
USPC 606/41

(57) ABSTRACT

A modular catheter comprises a tubular sheath module having one or more electrodes attached to the distal end of the tubular sheath and an elongate shape-imparting mechanism module. The shape-imparting mechanism module is removably received within a lumen defined by the tubular member. The tubular sheath module and the shape-imparting mechanism module are releasably connectable to a handle module. The handle comprises a plurality of wall members defining a cavity housing one or more modules releasably connected to the handle and accessible by at least partially separating the wall members. The tubular sheath, the shape-imparting mechanism module and the handle are replaceable independently and can be sterilized for subsequent use.

### CROSS-REFERENCE TO RELATED APPLICATIONS

This application is a national phase entry under 35 U.S.C. §371 of International Patent Application PCT/AU2011/001677, filed Dec. 23, 2011, designating the United States of America and published in English as International Patent Publication WO 2012/088564 A1 on Jul. 5, 2012, which claims the benefit under Article 8 of the Patent Cooperation Treaty and under 35 U.S.C. §119(e) to U.S. Provisional Patent Application Ser. No. 61/427,335, filed Dec. 27, 2010, the disclosure of each of which is hereby incorporated herein by this reference in its entirety.

### TECHNICAL FIELD

This specification relates to a catheter assembly. For example, this specification describes a reprocessable modular catheter assembly that can be disassembled and reassembled from reusable or interchangeable modules.

### BACKGROUND

Any discussion of the prior art throughout the specification should in no way be considered as an admission that such prior art is widely known or forms part of common general knowledge.

Electrophysiology catheters are commonly used in medical practice to examine and treat the heart. They may be inserted into the cardiovascular system of the patient through small punctures in the skin. They may then extend through a vein into the heart where they sense the electrical activity of the heart. Some of the electrophysiology catheters may be able to treat the heart by ablating the appropriate areas of the heart in case of certain types of aberrant electrical activity. Catheters generally include a tubular structure such as a plastic tube with one or more electrodes attached to the tip of the tube and a handle connected to the tubular structure. The electrodes are connected via electrical conductors to instruments such as a monitor or a stimulator.

During their use in medical procedures, catheters are exposed to biological fluids in the human body. Because catheters come in contact with these bodily fluids, they are commonly designed to be single-use devices to avoid the transfer of viruses or bacteria from one patient to another. Disposing of the catheters after each procedure leads to significant expenses to the patient and the healthcare system, as well as creates a substantial amount of medical waste. Catheters, particularly the handles and sheaths of catheters, are typically an expensive piece of equipment. They include expensive components and materials such as platinum-iridium electrodes, components used in the construction of the deflection mechanism, and proprietary electronics used in the handle for increased functionality. These valuable components are lost when the device is disposed after a single use. Because catheters are expensive and their use produces a large amount of waste, certain institutions sterilize or reprocess catheters for re-use. Reprocessing single-use devices reduces the cost of a procedure and minimizes medical waste generated.

However, as catheters are usually designed only for a single use, the reprocessed device may not function as intended. Often, one part of the reprocessed catheter is no longer functional, yet the entire device must be disposed.

In addition, catheters are typically fabricated as substantially integral devices where most components are joined together using permanent connections and seals. These types of catheters are difficult or impossible to resterilize reliably. In addition, the increasing complexity of electrophysiology catheters makes the re-sterilization process of catheters more difficult. Some of the delicate parts of the catheter may deteriorate during the re-sterilization process, which may lead to malfunction of the catheter. In some cases, small parts of the catheter may become detached when the catheter is reused having consequences on patient safety. Furthermore, small particles of biological matter may be trapped within the catheter even after the sterilization process.

### BRIEF SUMMARY

It is an object of the present invention to overcome or ameliorate at least one of the disadvantages of the prior art, or to provide a useful alternative.

According to one embodiment, there is provided a reusable modular catheter including: a tubular sheath having a proximal end and a distal end, the tubular sheath having one or more electrodes attached to the distal end of the tubular sheath, an elongate shape-imparting mechanism module having a proximal end and a distal end, the shape-imparting mechanism module received within a lumen defined by the tubular member such that the distal end of the shape-imparting mechanism module is substantially in register with the distal end of the tubular sheath, and a handle releasably connectable to the proximal end of the tubular sheath and the proximal end of the shape-imparting element, the handle comprising a plurality of wall members defining a cavity housing one or more modules releasably connected to the handle and accessible by at least partially separating the wall members.

One embodiment provides that the tubular sheath, the shape-imparting mechanism module and the handle are detachably connectable to one another. In addition, the tubular sheath, the shape-imparting mechanism module and the handle are replaceable independently of one another.

One embodiment provides that the one or more modules housed by the cavity comprises at least one electronic circuit board. The electronic circuit board is preferably arranged to perform signal processing, and it may be arranged to identify the modular catheter, and/or provide feedback to external devices.

According to one embodiment of the invention, the plurality of wall members are detachable from one another.

According to one embodiment of the invention, the tubular sheath, the shape-imparting mechanism module and the handle are sterilizable. The handle may also be sterilizable from within the cavity by accessing the cavity and removing any modules that are not sterilizable. The handle may further comprise one or more modules releasably attached to an outer surface of the handle. The one or more modules may be connected to the outer surface of the handle and may further be arranged to engage with at least one module housed by the cavity. According to one embodiment, the shape-imparting mechanism comprises a steering mechanism.

Reference throughout this specification to “one embodiment” or “an embodiment” or “some embodiments” means that a particular feature, structure or characteristic described in connection with the embodiment is included in at least one embodiment of the present invention. Thus, appearances of the phrases “in one embodiment” or “in an embodiment” or “in some embodiments” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

### BRIEF DESCRIPTION OF THE DRAWINGS

FIG. 1a shows modules of a modular catheter assembly detached from one another;

FIG. 1b shows a cross-sectional side view of a modular catheter when the modules have been assembled together;

FIG. 1c shows a side view of the modular catheter when it is assembled;

FIGS. 2a to 2d show different alternatives for the placement of electrodes at the distal end of the tubular sheath;

FIG. 3a shows modules of an embodiment of the modular catheter having a tubular sheath that is integrated with the catheter connector;

FIG. 3b shows another embodiment of the modular catheter having a combined steering mechanism and handle module and a tubular sheath that is integrated with the catheter connector; and

FIGS. 4a to 4c show an embodiment of the modular catheter having a sensor attached to the tip of the steering mechanism, and an electronic circuit board embedded in the handle of the catheter.

### DETAILED DESCRIPTION

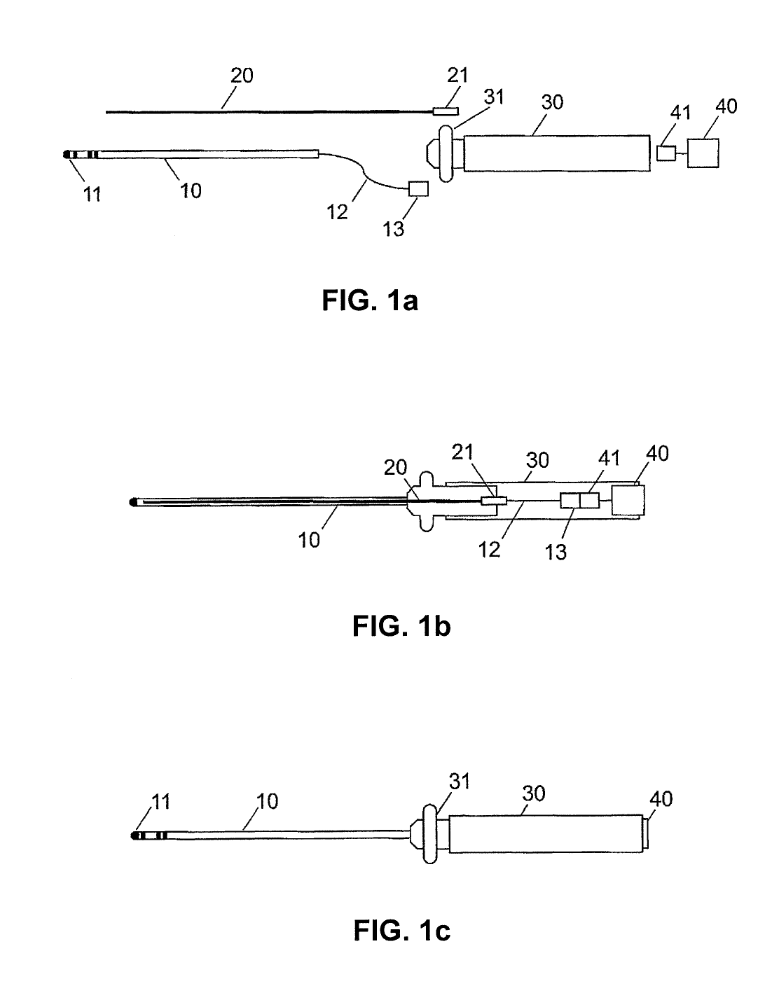

FIGS. 1a, 1b and 1c depict a modular catheter having a tubular electrode sheath 10, a detachable shape-imparting mechanism or a steering mechanism 20 and a detachable handle 30. The tubular electrode sheath 10 includes one or more electrodes attached to the tip, or the distal end, of the sheath 10. In this specification, the term “distal” refers generally to the direction that is furthest away from the user of the catheter. Respectively, the term “proximal” in this specification refers generally to the direction that is closest to the user when the catheter is in use.

In FIG. 1a, the modular components of the catheter are depicted separately when the components are detached from one another. The electrode sheath 10 is a tubular member that carries electrodes 11 at its distal end and wires 12 for conducting electrical signals from the electrodes to a connector element 13 at the proximal end of the sheath 10. The connector 13 may be any suitable form of electrical connector such as an electrical plug-type arrangement, a slip-ring type arrangement or a connector having a male and a female mating connector bodies. The handle module 30 also houses a deflection knob 31, which may move relative to the handle. The connector module 40 allows connection of the catheter to electrical instruments via a patient extension cable, for example, to a monitor, a stimulator or a source of energy such as an RF energy source used for ablation. The connector module 40 is further connected to an internal connector 41 to allow connection/disconnection with the connector wires 12 of the tubular sheath module 10.

The catheter also includes a shape-imparting mechanism in the form of a steering mechanism module 20. FIGS. 1a to 1c have a separate steering mechanism module 20 such as a stylet for guiding the catheter in the cardiovascular system of a patient when the catheter is in use. The steering mechanism module enables controlling the lateral displacement and subsequent radius of curvature of the distal end of the electrode sheath 10 to allow placement of the catheter tip at the desired location in the patient's cardiovascular system.

FIG. 1b shows a schematic cross-section of the catheter when the components of the modular catheter are assembled together. The steering mechanism module 20 is inserted inside a lumen defined by the tubular sheath 10 so that the distal end of the steering mechanism module is substantially in register with the distal tip of the tubular sheath 10. The steering mechanism module 20 is connected to the deflection knob 31 and/or the handle 30 via the coupling termination 21. The conductive wires 12 of the tubular sheath 10 lead to the connector 13, which engages with the rear connector 40 via an internal connector 41.

The electrode sheath 10 and the steering mechanism module 20 are each releasably connectable to the handle 30 independently of one another. However, it should be understood that, although the steering mechanism 20 and the electrode sheath 10 are described here as separate structures, they may also be affixed to one another as long as that unitary structure is then detachably connectable to the handle 30. The steering mechanism module 20 may consist of a wire, or a wire and a tube. When the coupling termination 21 is pulled, the distal end of the catheter will deflect and guide the distal end of the catheter. When the tubular sheath 10 and the steering mechanism module 20 are coupled to the deflection knob 31 and/or the handle 30, the relative movement of the steering mechanism 20 and the handle 30 causes lateral displacement of the distal tip of the catheter. The deflection knob 31 preferably comprises a pair of actuators or slides that are displaceable longitudinally along the handle 30. The proximal end of the steering mechanism unit 20 is connected to the actuators so that sliding the knob 31 along the handle, the tip or the distal end of the steering mechanism bends or deflects, thus guiding the catheter tip in a desired direction in use.

FIG. 1c shows the assembled catheter, with electrodes 11, electrode sheath 10, deflection knob 31, handle 30 and connector 40. The connector 40 at the proximal end of the handle may incorporate a slip ring arrangement or a similar connecting arrangement allowing rotation of the handle without rotating the cable connecting the catheter to a monitoring system (the cable and the monitoring system not shown in FIGS. 1a to 1c).

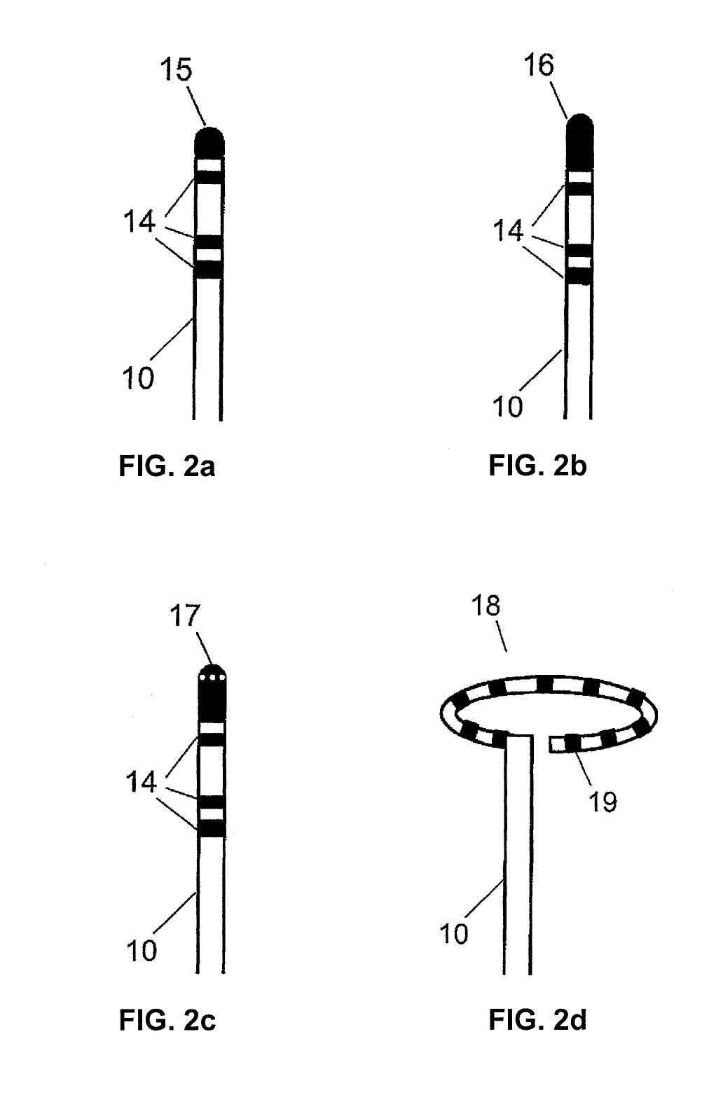

The modular catheter allows the selection of a tubular sheath 10 from a plurality of tubular sheaths, each having a different electrode configuration for different applications. FIGS. 2a to 2c show variants of the tubular sheath module 10, with a number of sensing electrodes 14, for different catheter types. These include catheters with a diagnostic catheter tip 15 (FIG. 2a), an ablation catheter tip 16 (FIG. 2b), an irrigated ablation catheter tip 17 (FIG. 2c), and a loop catheter with a loop-shaped electrode region 18 with multiple electrodes 19 (FIG. 2d).

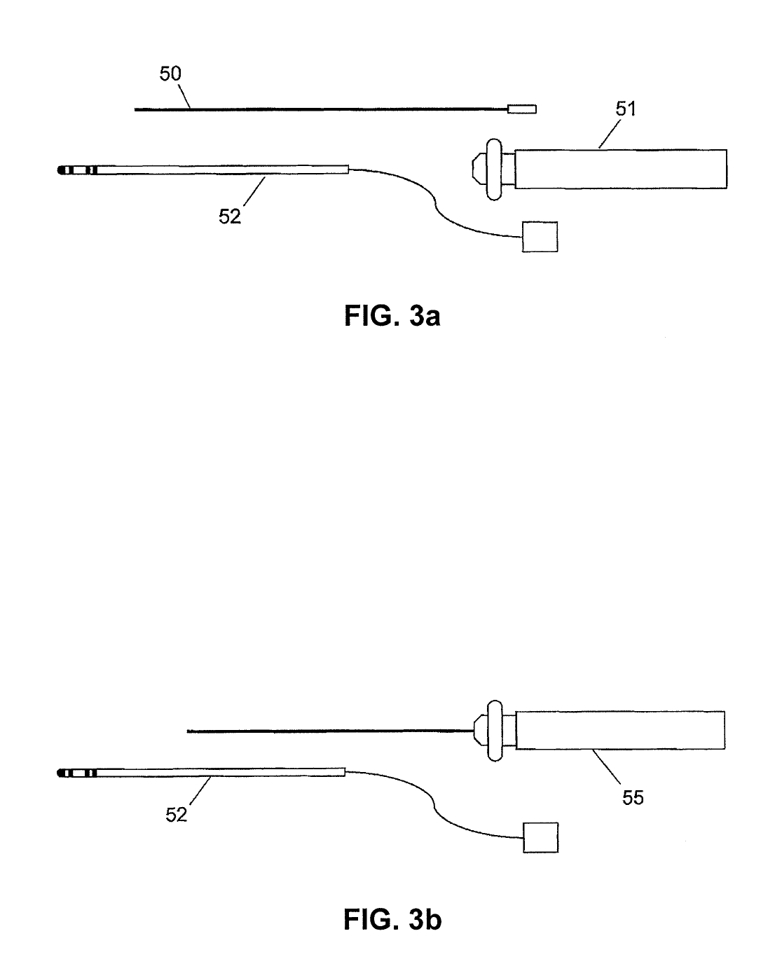

FIGS. 3a and 3b show variants of the modular catheter assembly with other module configurations. FIG. 3a shows the steering mechanism module 50, the handle module 51 and the combined tubular electrode sheath and connector module 52. In this example, the connector 40 may be placed at the proximal end of the handle, or the combined tubular electrode sheath and connector module 52 can be directly connected to a cable connecting the catheter to a monitoring system (not shown). FIG. 3b shows the combined electrode sheath and connector module 52, with a combined handle and steering mechanism module 50. In FIG. 3b, the handle and the steering mechanism are affixed together and the combined electrode sheath and connector module 52 can be removably attached to the handle or directly to a cable leading to a patient monitoring system. Other variants with two or more modules are possible. The tubular electrode sheath of FIGS. 3a and 3b may be chosen from a variety of electrode sheaths having different configurations of electrodes.

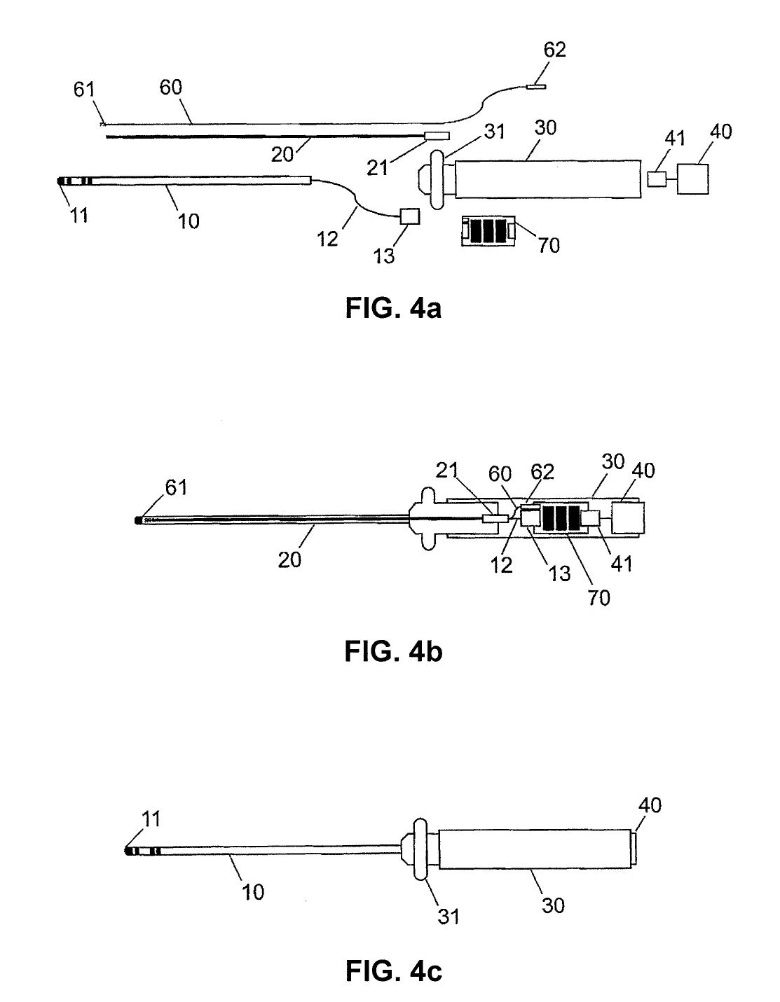

FIGS. 4a, 4b and 4c depict the modular catheter having an electronic circuit board located in the handle of the catheter.

In FIG. 4a, the modular components of the catheter are depicted separately when the components are detached from one another. Similarly to FIG. 1a, reference number 10 indicates the electrode sheath, which is a tubular member that carries electrodes 11 and wires 12 to conduct electrical signals from the electrodes to a connector element 13. The connector 13 is preferably a universal type connector to cater for the different ranges of electrode configurations shown in FIGS. 2a to 2d. The steering mechanism module 20 enables deflecting the tip, or the distal end, of the sheath 10 and it may consist of a wire, or a wire and a tube such as a stylet. The steering mechanism module 20 is inserted into the sheath 10 as seen in FIG. 4b and connected to the handle 30 via a termination 21 located at the proximal end of the steering mechanism module 20. Longitudinal movement of the termination 21 relative to the catheter sheath 10, which results in deflection of the catheter tip, is accomplished by suitable manipulation of the handle 30. The tubular sheath 10 is anchored to the control knob 31 of the handle 30 and the termination 21 is anchored either to the handle 30 or to the control knob 31 and the handle 30 so that movement of the termination 21 deflects the tip of the catheter. Control knob 31 and the tubular sheath 10 move relative to the handle 30 so that steering mechanism module imparts an appropriate deflection to the tubular sheath 10.

As seen in FIGS. 4a and 4b, the modular catheter may also include a sensor 61 with an electrical conductor or conductors 60 and a suitable internal connector 62. The sensor 61 may be used for such applications as sensing temperature, sensing contact forces, and/or determining the position of the tip. The sensor 61 is arranged within the sheath 10 and connected via the conductor 60 and the connector 62 to an electronic board 70. The electronic circuit board 70 may be used, for example, for identifying the device, processing signals from the electrode sheath 10 and/or sensor 61, and providing feedback to external devices such as imaging monitors, computers or similar. In FIG. 4b, the connector 13 of the tubular sheath 10 is connected to the electronic board 70, which is further connected via the internal connector 41 and the rear connector 40 to the attached computer or monitoring system. The signals from the electronic board 70 are carried in a suitable form understandable by the external device.

The handle 30 comprises one or more wall members that form a cavity within the handle. The wall members are preferably a pair of shells that can be connected together by any suitable connector pairs. The connector may be snap-lock-type connectors, complimentary pins and sockets, or a sliding connector arrangement. In addition, the shells can also be connected by a hinge on one side and a pair of releasably connected connectors so that the cavity inside the handle can be accessed by opening the shells or partly separating the shells from one another. The handle is preferably made of a durable and rigid material and ergonomically formed so as to allow the user to more easily manipulate the catheter. The electronic board 70 is removably connected to the handle by suitable connectors that allow easy connection/disconnection of the circuit board 70 from the handle. Because electronic circuit boards embedded in catheter handles contribute to the cost of a catheter, it is advantageous to have a detachable circuit board so that the circuit board may be reused if some other part of the handle cannot be used further or replaced by a new circuit board in case the old one stops functioning properly. In addition, the circuit board may be removed if the handle is being sterilized. The handle may have different variations of the types of controls or shape for any of the modular catheters described in this specification. The user may choose the type of control handle that he wishes to use for a particular medical procedure.

FIG. 4c shows the completed catheter, with electrodes 11, electrode sheath 10, deflection knob 31, handle 30 and connector 40.

After use, once the catheter has been removed from the patient's body, the catheter may be disassembled by disconnecting the connector from the handle and decoupling the coupling member from the handle 30. The tubular sheath 10, having been in contact with the patient, may be disposed of and replaced by a new one or alternatively, sterilized for subsequent use. The handle 30 may be detached from the tubular sheath 10 and the steering mechanism 20 and reused and sterilized, if necessary, without detaching the components such as the circuit board 70 and the connectors 41 and 40 from the handle. Alternatively, the handle may be opened and the components inside the handle may be removed for the sterilization process to ensure a better sterilization result. Furthermore, the steering mechanism module 20 can be detached from the handle and reused after sterilization, if necessary.

It is an advantage of the modular catheter that because the steering mechanism module is detachable from the tubular sheath 10 and the handle 30, it may comprise a variety of different solutions. The steering mechanism module 20 may be a stylet and it may impart a shape on the tubular sheath 10. The shape may be a pre-determined shape in the form of a curve or a loop, whereby the shape of the tubular sheath may be alternated by changing the steering mechanism module to another one having a different shape. In addition, the shape of the steering mechanism may be controlled by alternating the radius of curvature of the shape or changing the shape to another shape or curve.

It is a further advantage of the modular catheter that any module of the catheter may be disposed of or sterilized independently from the others. This means that units that are particularly durable in use and/or sterilization such as the steering mechanism module 20 or the handle 30 may be reused for any number of times. Modules that may deteriorate quicker, such as the modular sheath 10, may be reused as many times as they endure the sterilization process and then discarded independently of the other modules of the catheter. In addition, if any module stops functioning properly, it may be replaced by a new one without discarding the other modules that still function as intended. This reduces the cost of a catheter significantly over time.

The modular structure of the catheter provides easy connection or disconnection of the modules and allows a variety of modules having different functions to be used together or separately as needed in specific medical procedures. The user can choose the type of sheath, steering mechanism or handle he wishes to use for a particular medical procedure. The user can further choose which modules of the catheter he wants to have reprocessed and sterilized for subsequent use. The modularity of the catheter also allows remanufacturing of cardiac catheters by reusing some or all of the modules of catheters, and using them to manufacture another catheter, in particular, another type of catheter.

Furthermore, reprocessing of the modular catheter is easier and more reliable as the modules can be detached from one another in order to ensure that biological matter is not trapped on the modules and particularly, in the connection points of the modules. Moreover, the modularity of the handle allows more reliable sterilization of the handle, especially if the handle is opened and sterilized from inside the handle as well. Because the handle consists of shell-like components, there is no need for watertight seals, which makes the manufacturing process less complex.

It is a further advantage of the modular catheter that a device that is made of reusable modules is better economically, and better for the environment. Furthermore, by maximizing the life of the device, the cost and environmental benefits are also maximized. These benefits are enhanced by having a catheter constructed with user-changeable modules.

A catheter specifically designed to be reprocessed would lead to a safer, more reliable, reprocessed catheter. This catheter would also lead to savings in cost per procedure, as expensive parts could be reused, reducing environmental impact and generating minimal medical waste.

Reference throughout this specification to “one embodiment,” “some embodiments,” or “an embodiment” means that a particular feature, structure or characteristic described in connection with the embodiment is included in at least one embodiment of the present invention. Thus, appearances of the phrases “in one embodiment,” “in some embodiments,” or “in an embodiment” in various places throughout this specification are not necessarily all referring to the same embodiment, but may. Furthermore, the particular features, structures or characteristics may be combined in any suitable manner, as would be apparent to one of ordinarily skill in the art from this disclosure, in one or more embodiments.

As used herein, unless otherwise specified, the use of ordinal adjectives “first,” “second,” “third,” etc., to describe a common object, merely indicate that different instances of like objects are referred to, and are not intended to imply that the objects so described must be in a given sequence, either temporally, spatially, in ranking, or in any other manner.

In the claims below and the description herein, any one of the terms “comprising,” “comprised of,” or “which comprises” is an open term that means including at least the elements/features that follow, but not excluding others. Thus, the term “comprising,” when used in the claims, should not be interpreted as being limitative to the means or elements or steps listed thereafter. For example, the scope of the expression “a device comprising A and B” should not be limited to devices consisting only of elements A and B. Any one of the terms “including,” or “which includes,” or “that includes,” as used herein, is also an open term that also means including at least the elements/features that follow the term, but not excluding others. Thus, “including” is synonymous with and means “comprising.”

It should be appreciated that in the above description of exemplary embodiments of the invention, various features of the invention are sometimes grouped together in a single embodiment, figure, or description thereof, for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment Thus, the claims following the Detailed Description are hereby expressly incorporated into this Detailed Description, with each claim standing on its own as a separate embodiment of this invention.

Furthermore, while some embodiments described herein include some, but not other, features included in other embodiments, combination of features of different embodiments are meant to be within the scope of the invention and form different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term “coupled,” when used in the claims, should not be interpreted as being limited to direct connections only. The terms “coupled” and “connected,” along with their derivatives, may be used. It should be understood that these terms are not intended as synonyms for each other. Thus, the scope of the expression “a device A coupled to a device B” should not be limited to devices or systems wherein an output of device A is directly connected to an input of device B. It means that there exists a path between an output of A and an input of B, which may be a path including other devices or means. “Coupled” may mean that two or more elements are either in direct physical or electrical contact, or that two or more elements are not in direct contact with each other but yet still cooperate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the invention.

It will be appreciated by persons skilled in the art that numerous variations and/or modifications may be made to the disclosure as shown in the specific embodiments without departing from the scope of the disclosure as broadly described. The present embodiments are, therefore, to be considered in all respects as illustrative and not restrictive.

1. A reusable modular catheter, comprising:
a tubular sheath having a proximal end and a distal end, the tubular sheath having one or more electrodes attached to the distal end of the tubular sheath;
an elongate shape-imparting mechanism module having a proximal end and a distal end, the shape-imparting mechanism module including a shape-imparting element received within a lumen defined by the tubular member such that the distal end of the shape-imparting mechanism module is substantially in register with the distal end of the tubular sheath; and
a handle releasably connectable to the proximal end of the tubular sheath and the proximal end of the shape-imparting element, the handle comprising a plurality of wall members defining a cavity housing one or more modules releasably connected to the handle and accessible by at least partially separating the wall members.

2. The reusable modular catheter of claim 1, wherein the tubular sheath, the shape-imparting mechanism module and the handle are detachably connectable to one another.

3. The reusable modular catheter of claim 1, wherein the tubular sheath, the shape-imparting mechanism module and the handle are replaceable independently of one another.

4. The reusable modular catheter of claim 1, wherein the one or more modules housed by the cavity comprises at least one electronic circuit board.

5. The reusable modular catheter of claim 4, wherein the electronic circuit board is configured to perform signal processing.

6. The reusable modular catheter of claim 4, wherein the electronic circuit board is configured to identify the modular catheter.

7. The reusable modular catheter of claim 4, wherein the electronic circuit board is configured to provide feedback to external devices.

8. The reusable modular catheter of claim 1, wherein the plurality of wall members are detachable from one another.

9. The reusable modular catheter of claim 1, wherein the tubular sheath, the shape-imparting mechanism module and the handle are sterilizable.

10. The reusable modular catheter of claim 9, wherein the handle is sterilizable within the cavity by accessing the cavity and removing any modules that are not sterilizable.

11. The reusable modular catheter of claim 1, wherein the shape-imparting mechanism module comprises a steering mechanism.

12. The reusable modular catheter of claim 1, wherein the handle further comprises one or more modules releasably attached to an outer surface of the handle.

13. The reusable modular catheter of claim 12, wherein the one or more modules connected to the outer surface of the handle are arranged to engage with at least one module housed by the cavity.

14. The reusable modular catheter of claim 4, further comprising a sensor arranged within the tubular sheath and connected to the at least one electronic circuit board by a conductor.

15. The reusable modular catheter of claim 14, wherein the sensor is configured for sensing at least one of temperature, contact forces, and a position of the distal end of the tubular sheath.

16. A reusable modular catheter, comprising:
a tubular sheath including at least one lumen, the tubular sheath having a proximal end and a distal end, the tubular sheath having one or more electrodes at the distal end of the tubular sheath;
a shape-imparting mechanism module including a shape-imparting element receivable within the at least one lumen of the tubular sheath such that a distal end of the shape-imparting element is substantially in register with the distal end of the tubular sheath; and
a handle releasably connectable to the proximal end of the tubular sheath and the proximal end of the shape-imparting mechanism module, the handle comprising a plurality of wall members defining a cavity housing one or more modules releasably connected to the handle and accessible by at least partially separating the wall members, the one or more modules comprising an electronic module including at least one electronic circuit board.

17. The reusable modular catheter of claim 16, wherein the tubular sheath, the shape-imparting mechanism module and the handle are detachably connectable to one another, and wherein the tubular sheath, the shape-imparting mechanism module and the handle are replaceable independently of one another.

18. A method of using a reusable modular catheter, comprising:
providing a reusable modular catheter including:
a tubular sheath having a proximal end and a distal end, the tubular sheath having one or more electrodes attached to the distal end of the tubular sheath;
an elongate shape-imparting mechanism module having a proximal end and a distal end, the shape-imparting mechanism module including a shape-imparting element received within a lumen defined by the tubular member such that the distal end of the shape-imparting mechanism module is substantially in register with the distal end of the tubular sheath; and
a handle releasably connectable to the proximal end of the tubular sheath and the proximal end of the shape-imparting element, the handle comprising a plurality of wall members defining a cavity housing one or more modules releasably connected to the handle and accessible by at least partially separating the wall members;
using the reusable modular catheter to treat a patient;
separating the tubular sheath, the elongate shape-imparting mechanism module, and the handle from one another and sterilizing each of the tubular sheath, the elongate shape-imparting mechanism module, and the handle; and
reusing each of the tubular sheath, the elongate shape-imparting mechanism module, and the handle to treat another patient.

19. The method of claim 18, wherein reusing each of the tubular sheath, the elongate shape-imparting mechanism module, and the handle to treat another patient comprises reassembling each of the tubular sheath, the elongate shape-imparting mechanism module, and the handle with one another to form the reusable modular catheter, and using the same reusable modular catheter to treat the patient and the another patient.

20. The method of claim 18, wherein the one or more modules housed by the cavity comprises at least one electronic circuit board, and wherein separating the tubular sheath, the elongate shape-imparting mechanism module, and the handle from one another comprises removing the at least one electronic circuit board from the handle.
